SEQUENCE LISTING (2 sequences)

SEQ ID NO: 1 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 2 - protein, 172 aa, Artificial Sequence
  Recombinant IFNtau based on Ovis aries sequence
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

(19) United States
(12) Patent Application Publication
(10) Pub. No.: US 2009/0035273 A1
(43) Pub. Date: Feb. 5, 2009

(54) Combination treatment method with interferon-tau

(75) Inventor: Chih-Ping Liu, Pleasanton, CA (US)

Correspondence Address: PERKINS COIE LLP, P.O. BOX 1208, SEATTLE, WA, 98111-1208, US

(73) Assignee: Pepgen Corporation, Alameda, CA (US)

(21) Appl. No.: 11/893,768

(22) Filed: Aug. 16, 2007

Related U.S. Application Data
(60) Provisional application No. 60/838,722, filed on Aug. 18, 2006.

(51) Int. Cl. A61K 38/21; A61P 31/12; A61P 35/00

(52) U.S. Cl. 424/85.6; 424/85.7

(57) ABSTRACT

A method of treating conditions responsive to therapy with interferon-alpha or interferon-beta is provided, where the dose of interferon-alpha or interferon-beta is reduced and a dose of interferon-tau is additionally administered. The method results in efficacious therapy with a reduction in unwanted adverse events.

### CROSS-REFERENCE TO RELATED APPLICATIONS

This application claims the benefit of U.S. Provisional Application No. 60/838,722, filed Aug. 18, 2006, which is incorporated herein by reference in its entirety.

### TECHNICAL FIELD

The subject matter described herein relates to a method of treating viral infections, and other disorders, using an interferon therapy. More particularly, the subject matter relates to an interferon treatment method, where a reduced dose of a type I, recombinant interferon other than interferon-tau, such as interferon-alpha or interferon-beta, is provided in conjunction with a dose of interferon-tau.

### BACKGROUND

Several different types of interferon are now approved for use in humans, and interferon therapy is used, often in combination with other drugs or treatments, for treating viral infections, certain cancers, and certain autoimmune conditions. Several different forms of interferon alpha, including interferon-alpha-2a, interferon-alpha-2b, and interferon-alfacon-1, are approved for the treatment of viral hepatitis. More recently, pegylated forms of interferon-alpha, in which polyethylene glycol is attached to the polypeptide to increase its circulation time in the body, have been approved for use. A pegylated interferon-alpha-2b and a pegylated interferon-alpha-2a are commercially available for use. The pegylated form is injected once weekly, rather than three times per week for conventional interferon-alpha. Interferon-beta, and specifically interferon-beta 1a and interferon-beta 1b, is used in the treatment and control of the autoimmune disorder multiple sclerosis.

When used in systemic therapy, interferon-alpha, interferon-beta, and interferon-gamma, are typically administered by injection intramuscularly or subcutaneously. Frequent side-effects are flu-like symptoms: increased body temperature, ill feeling, fatigue, headache, muscle pain, and convulsion. Erythema, pain and hardness on the spot of injection are also frequently observed. Often patients report feeling worse during therapy than before. As a result, treatment is discontinued and no resolution or improvement of the disease occurs, ultimately leading to a worse condition for the patient. When therapy is terminated because the side effects are too severe, the virus or cancer remaining in the infected patient may develop resistance to the therapy, rendering subsequent treatment ineffective. Thus, patient non-compliance and the discontinuation of therapy due to side effects remains a significant problem to the field of antiviral therapy.

One approach to reducing the flu-like symptoms of headache and fatigue associated with interferon therapy is to reduce the dose of interferon, which reduces the therapeutic benefit. Another way to reduce the side effects is to utilize a secondary therapy, such as an analgesic or an antipyretic, that treats the unwanted side effect. However, this complicates the treatment and can lead to unwanted drug interactions or still other unwanted effects, such as gastrointestinal discomfort.

A need remains to improve treatment with interferon therapy in general, to increase patient compliance with the necessary treatment protocols, and to improve the likelihood of successful treatment and resultant disease eradication.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a method for attenuating side effects resulting from treatment with an interferon-alpha or an interferon-beta at a recommended dose is provided. The method involves administering via injection the interferon-alpha or the interferon-beta in an amount less than the recommended dose for treatment of a disorder, said amount being insufficient for treatment of the disorder and, optionally, causing fewer side effects than the recommended dose for treatment; and administering by mouth interferon-tau in an amount effective alone or in combination with the interferon-alpha or interferon-beta for treatment of the disorder.

In one embodiment, interferon-alpha2b is administered via injection. In another embodiment, the administered interferon-alpha2b is administered for treatment of a disorder selected from the group consisting of chronic hepatitis C, chronic hepatitis B, malignant myeloma, hairy cell leukemia, renal cell carcinoma, and Kaposi's sarcoma.

In another embodiment, pegylated interferon-alpha2b is administered, for treatment of, for example, hepatitis C.

In another embodiment, interferon-alpha2a is administered in a dose reduced amount, for treatment of, for example, chronic hepatitis C, chronic hepatitis B, malignant myeloma, hairy cell leukemia, renal cell carcinoma, or Kaposi's sarcoma.

In another embodiment, pegylated interferon-alpha2a is administered, for treatment of any of the aforementioned conditions.

In still another embodiment, a dose of interferon-beta1a or interferon-beta1b is delivered in an amount less than the labeled recommended amount, for treatment of, for example, relapsing forms of multiple sclerosis.

In yet another embodiment, an amount of consensus interferon-alpha is administered to a person diagnosed with hepatitis C, the amount being less than the recommended dose.

In another aspect, an improvement in a method of treating a patient suffering from a disorder indicated for treatment with an interferon-alpha or an interferon-beta at a recommended dose is provided. The improvement comprises reducing the dose of interferon-alpha or interferon-beta to less than the recommended dose and, optionally, in an amount sufficient to reduce adverse events, and co-administering a dose interferon-tau, wherein the dose of interferon-alpha or interferon-beta and the dose of interferon-tau provide a combined total dose that is therapeutically effective for the disorder.

In one embodiment, co-administering comprises co-administering interferon-tau to the oropharyngeal region.

In another embodiment, co-administering comprises co-administering interferon-tau orally to the gastrointestinal tract.

In yet another embodiment, reducing comprises reducing the dose and administering the reduced dose via injection.

In a related aspect, use of interferon-tau for the manufacture of a medicament for administration in combination with an interferon-alpha or an interferon-beta at a dosage less than recommended for treatment is provided.

In some embodiments, the administering of interferon-alpha or interferon-beta is by injection and said administering of interferon-tau is by mouth.

In some embodiments, the interferon-alpha or interferon-beta is interferon-alpha2b. In other embodiments, the interferon-alpha or interferon-beta is interferon-alpha2a. In other embodiments, the interferon-alpha or interferon-beta is consensus interferon-alpha. In other embodiments, the interferon-alpha or interferon-beta is interferon-beta1b. In other embodiments, the interferon-alpha or interferon-beta is interferon-beta1a.

In addition to the exemplary aspects and embodiments described above, further aspects and embodiments will become apparent by reference to the drawings and by study of the following descriptions.

### BRIEF DESCRIPTION OF THE SEQUENCES

SEQ ID NO:1 corresponds to an amino acid sequence of mature ovine interferon-τ (IFNτ; oTP-1; GenBank Accession No. Y00287; PID g1358).

SEQ ID NO:2 corresponds to an amino acid sequence of mature ovine IFNτ, where the amino acid residues at positions 5 and 6 of the sequence are modified relative to the sequence of SEQ ID NO:1.

### DETAILED DESCRIPTION

A method for improving treatment of viral infections, cancers, and autoimmune disorders with type I interferons is provided. In the method, the afflicted patient is treated with a first type I interferon, wherein the first type I interferon is a type I interferon other than interferon-tau, at a dose less than the recommended dose. In conjunction with the reduced dose of the first type I interferon, the patient is treated with a dose of interferon-tau. Together the combined interferon dose, i.e., the dose of the type I interferon other than interferon-tau plus the dose of interferon-tau, is sufficient to provide a therapeutic effect yet, due to the reduced dose of the first type I interferon, results in a reduction or absence of unwanted side effects:

The recommended dose generally corresponds to the dose described on the package insert for treatment of a particular condition. Such doses are typically approved by a regulatory agency, for example, the U.S. Food and Drug Administration. In some cases, when the interferon is being used “off-label”, the recommended dose is the dose commonly used by trained medical providers. In the method, the recommended dose of the type I interferon other than interferon-tau is reduced to a dose level that permits efficacious treatment, and optionally provides a desirable reduction in unwanted side effects.

It will be appreciated that while certain side effects are common to the population of patients treated with any particular interferon, the degree of discomfort and the actual side effects observed will vary from patient to patient, depending in part on the overall health of the patient and the indication being treated. The method described herein provides a means to tailor treatment for individual patients, by selecting a reduction in recommended dose for each individual patient in order to attenuate or inhibit certain side effects, yet provide efficacious therapy.

In the method, the first interferon administered is any type I interferon other than interferon-tau. Generally, this first type I interferon is a type I interferon that occurs in humans, irrespective of the polypeptide sequence actually administered. For example, interferon-alpha is endogenous to humans, and the method contemplates administration of an interferon-alpha to the patient, wherein the interferon-alpha can have, e.g., a human sequence of amino acids, a murine sequence of amino acids, a humanized sequence of amino acids, a consensus sequence of amino acids, or a sequence of amino acids from any other species or combination of species.

In preferred embodiments, the type I interferon is an interferon-alpha or an interferon-beta. There are thirteen different isoforms of interferon-alpha, interferon-alpha 1, 2, 4, 5, 6, 7, 8, 10, 13, 14, 16, 17, and 21. A single interferon-beta protein has been identified. Other type I interferons include interferon-omega, interferon-epsilon, and interferon-kappa. The type I interferons bind to a specific cell surface receptor complex known as interferon-alpha receptor consisting of interferon-alpha receptor 1 and interferon-alpha receptor 2 chains.

A dose reduction of the endogenous type I interferon can range from a reduction of between about 10-90% of the recommended dose. In certain embodiments, the dose of the type I interferon is reduced by 25% of the recommended dose, or by 30% of the recommended dose, or by 40% of the recommended dose, or by 50% of the recommended dose, or by 60% of the recommended dose, or by 70% of the recommended dose, or by 75% of the recommended dose, or even by 80% of the recommended dose that is indicated for the condition being treated.

The interferon-tau used in the treatment method refers to any one of a family of interferon-tau proteins having at least about 70% amino acid homology, preferably at least about 80% amino acid identity, more preferably at least about 90% sequence identity, and even 95% sequence identity, to a known interferon-tau sequence (e.g., Ott, et al., J. Interferon Res., 11:357 (1991); Helmer, et al., J. Reprod. Fert., 79:83 (1987); Imakawa, et al., Mol. Endocrinol, 3:127 (1989); Whaley, et al., J. Biol. Chem., 269:10846 (1994); Bazer, et al., WO 94/10313 (1994)). Exemplary levels of sequence identity include 90%, 91%, 92%, 93%, 94%, 95%, 96%, 97%, 98%, and 99%. Interferon-tau sequences have been identified in various ruminant species, including but not limited to, cow (Bovine sp., Helmer S. D., J. Reprod. Fert., 79:83 (1987); Imakawa, K., Mol. Endocrinol., 119:532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of interferon-tau for many of these species are reported in public databases and/or in the literature (see, for example, Roberts, R. M. et al., J. Interferon and Cytokine Res., 18:805 (1998), Leaman D. W. et al., J. Interferon Res., 12:1 (1993), Ryan, A. M. et al., Anim. Genet., 34:9 (1996)).

In a preferred embodiment, an ovine interferon-tau protein is used in the method. An exemplary ovine interferon-tau proteins is identified herein as SEQ ID NO:1. The preferred ovine interferon-tau is one having at least about 70%, more preferably 80%, still more preferably at least about 90% sequence identity, and even 95% sequence identity to SEQ ID NO:1. Exemplary levels of sequence identity include 90%, 91%, 92%, 93%, 94%, 95%, 96%, 97%, 98%, and 99%. Conservative amino acid substitutions and alterations such as neutral amino acid substitutions that do not significantly affect the activity of the protein can be made, and an example of such a modified ovine interferon-tau protein is identified herein as SEQ ID NO:2. Sequence homology is determined, for example, by a strict amino acid comparison or using one of the many programs commercially available.

Recombinant production of interferon-tau is described in both the scientific literature (Ott, et al., J. Interferon Cytokine Res., 11:357-364 (1991); Soos, J. M. et al., J. Immunol., 155:2747 (1995)) and the patent literature (WO/94/10313; US 2003/0049277), and the description of IFNτ production in these documents is incorporated by reference herein.

The treatment method will now be illustrated by way of specific examples where a type I interferon other than interferon-tau is administered to a patient at less than the recommended dose, in combination with a dose of interferon-tau, to achieve efficacious therapy with, optionally, a reduction in side effects.

### Interferon-alpha2b

In one embodiment, a combination therapy comprised of interferon-alpha2b and interferon-tau is provided. Interferon-alpha2b (Intron® A) is indicated for the treatment viral infections and certain cancers. In particular, the recombinant protein is approved for use in the treatment of patients with chronic hepatitis B virus infection, chronic hepatitis C virus infection, hairy cell leukemia, malignant melanoma, follicular (non-Hodgkin's) lymphoma, condylomata acuminate, and AlDS-relaed Kaposi's sarcoma.

The recommended dose of interferon-alpha 2b for treatment of hairy cell leukemia is 2×10⁶ IU/m² administered intramuscularly or subcutaneously three times a week for up to six months; malignant melanoma is 20×10⁶ IU/m² as an intravenous infusion over 20 minutes, 5 consecutive days per week, for 4 weeks; for follicular lymphoma is 5×10⁶ IU/m² administered subcutaneously three times a week for up to 18 months; condylomata acuminate is 1×10⁶ IU per lesion, the lesions injected three time weekly on alternate days for three weeks; for AIDS-related Kaposi's sarcoma is 30×10⁶ IU/m²/dose administered intramuscularly or subcutaneously three times a week until disease progression or maximal response has been achieved after 16 weeks of treatment; for chronic hepatitis C is 3×10⁶ IU administered intramuscularly or subcutaneously three times a week for at least 16 weeks or longer if the patient is responsive, as measured by normalization of blood levels of alanine aminotransferase (ALT); for chronic hepatitis B is 30-35×10⁶ IU administered intramuscularly or subcutaneously, either as a 5×10⁶ IU daily dose or at 10×10⁶ IU dose three times a week for 16 weeks.

The present method contemplates administering interferon-alpha 2b at a dose less than one of the recommended doses recited above for treatment of a particular disorder and additionally administering interferon-tau at a therapeutic or subtherapeutic dose. The combined treatment regimen provides efficacious therapy for treatment of the disorder but with a reduction in the unwanted side effects. Exemplary treatment regimens for chronic hepatitis C and malignant myeloma are given in Examples 1-2.

### Interferon-alpha2a

In another embodiment, a combination therapy comprised of a less than recommended dose of interferon-alpha2a and of interferon-tau is provided. Interferon-alpha2a (Roferon®) is indicated for the treatment of chronic hepatitis C, hairy cell leukemia, and AIDS-related Kaposi's sarcoma, as well as chronic phase Philadelphia chromosome positive chronic myelogenous leukemia. Unwanted adverse events during treatment at the recommended dosages of interferon-alpha2a include neutropenia, severe or life-threatening anemia, leucopenia and thrombocytopenia. In patients with hairy cell leukemia, increases in serum phosphorus and serum uric acid are observed.

The specific activity of the commercially-available protein is 2.7×10⁸ IU/mg, thus, the unit and multidose vials which range from 3×10⁶ IU to 36×10⁶ IU correspond to 11.1-133.3 μg/mL.

The recommended dosing regimens for the noted indications for interferon-alpha2a are as follows. For chronic hepatitis C, the recommended dosage is 3×10⁶ IU three times a week administered subcutaneously or intramuscularly for 18 months (48-52 weeks). An alternative recommended dosing regimen is 6×10⁶ IU three times a week for the first three months (12 weeks) followed by 3×10⁶ IU three times a week for 9 months (36 weeks). For chronic myelogenous leukemia, the recommended dosage is 9×10⁶ IU daily administered as a subcutaneous or intramuscular injection. In some cases, a dose escalation over the first week of treatment is recommended, from 3×10⁶ IU daily for 3 days to 6×10⁶ IU daily for 3 to the target dose of 9×10⁶ IU daily for the duration of treatment. For hairy cell leukemia, an induction dose of 3×10⁶ IU daily for 16-24 weeks, administered as a subcutaneous or intramuscular injection is recommended. A maintenance dose of 3×10⁶ IU three times a week is then given. For AIDS-related Kaposi's sarcoma, a dose of 36×10⁶ IU daily for 10-12 weeks, administered as a subcutaneous or intramuscular injection is recommended. An escalating dose schedule of 3×10⁶ IU, 9×10⁶ IU, and 18×10⁶ IU each daily for three days followed by 36×10⁶ IU daily for the remainder of the 10-12 week induction period is also recommended for some patients experiencing acute toxicity.

According, the method described herein contemplates delivering to a patient suffering from hepatitis C a dose of less than the recommended dosage of 3×10⁶ IU three times a week, or a dose of less than the recommended doses on the alternative recommended dosing regimen in combination with a dose of interferon-tau. The method also contemplates treating chronic myelogenous leukemia in a subject by administering a dose less than the recommended dosage of 9×10⁶ IU daily in combination with a oral dose of interferon-tau. Alternatively, a chronic myelogenous leukemia patient can be treated by providing a dose escalation schedule where less than the recommended dose of 3×10⁶ IU is given daily for 3 days, and then less than the recommended dose of 6×10⁶ IU is given daily for 3, followed by less than the recommended dose of 9×10⁶ IU daily for the duration of treatment, in combination with a dose of interferon-tau given orally. The method also contemplates treating a patient diagnosed with hairy cell leukemia, by selecting a dose that is less than the recommended dose of 3×10⁶ IU daily, and administering the selected dose for the recommended period of treatment of 16-24 weeks, and also administering to the patient an oral dose of interferon-tau. The method also contemplates treating AIDS-related Kaposi's sarcoma, by providing a dose of less than the recommended dose of 36×10⁶ IU interferon-alpha2a daily for about the recommended period of time and additionally providing to the patient a dose of interferon-tau. Example 3 sets forth a working example of treatment of a patient diagnosed with Kapsoi's sarcoma.

### Pegylated-interferon-alpha2a

Interferon-alpha2a covalently linked to a single branched bis-monomethoxy polyethylene glycol (PEG) chain of approximately 40,000 Daltons is commercially available as “peginterferon alfa-2a” under the trade designation Pegasys®. The PEG moiety is linked at a single site to the interferon-alpha protein via a stable amide bond to lysine. Peginterferon alfa-2a is indicated for the treatment of adults with chronic hepatitis C who have compensated liver disease and have not been previously treated with interferon alpha. The recommended label dose is 180 μg (1.0 mL) once weekly for 48 weeks by subcutaneous administration in the abdomen or thigh.

According to the product label of Pegasys®, when dose modification is required for moderate to severe adverse reactions (clinical and/or laboratory), initial dose reduction to 135 μg (0.75 mL) is generally adequate. However, in some cases, dose reduction to 90 μg (0.5 mL) may be needed. Following improvement of the adverse reaction, re-escalation of the dose may be considered. Dose reduction to 135 μg is recommended if the neutrophil count is less than 750 cells/mm³. For patients with absolute neutrophil count (ANC) values below 500 cells/mm³, treatment should be suspended until ANC values return to more than 1000 cells/mm³. Therapy should initially be reinstituted at 90 μg Pegasys®, and the neutrophil count monitored. Dose reduction to 90 μg Pegasys® is recommended if the platelet count is less than 50,000 cells/mm³. Cessation of therapy is recommended when platelet count is below 25,000 cells/mm³.

Accordingly, the present method contemplates a method wherein pegylated-interferon-alpha2a is given at less than the recommended label dose, for example at one of the recommended reduced dosages and the treatment is supplemented by administration orally of interferon-tau. The total combined interferon dose, i.e., dose of pegylated-interferon-alpha2a and interferon-tau, is sufficient for treatment of hepatitis C, but does not result in adverse events. An exemplary treatment regimen is described in Example 4.

### Pegylated-interferon-alpha2b

In yet another embodiment, the method provides a treatment regiment using a reduced dose of pegylated-interferon-alpha2b (PEG-Intron®) and interferon-tau. Peginterferon alfa-2b is a covalent conjugate of recombinant interferon-alpha2b with monomethoxy polyethylene glycol (PEG) of 12,000 Daltons. The specific activity of the pegylated interferon-alpha2b is approximately 0.7×10⁸ IU/mg protein. Peginterferon alfa-2b is indicated for the treatment of chronic hepatitis C in patients not previously treated with interferon-alpha. Nearly all patients treated with peginterferon alfa-2b experience one or more adverse events, ranging from flu-like symptoms, injection site inflammation, psychiatric adverse events including depression and suicidal behavior.

Peginterferon alfa-2b is recommended for subcutaneous administration once weekly for one year. The recommended dose is based on the patients weight, as follows. For a weight of between 3745 kg, a dose of 40 μg; between 46-56 kg, a dose of 50 μg; between 57-72 kg, a dose of 64 μg; between 73-88 kg, a dose of 80 μg; between 89-106 kg, a dose of 96 μg; between 107-136 kg, a dose of 120 μg; between 137-160 kg, a dose of 150 μg.

The method contemplates treating a hepatitis C patient with a dose of peginterferon alfa-2b at less than the recommended dose for the patients weight and supplementing the treatment with an oral dose of interferon-tau.

### Interferon-alfacon-1

In another embodiment, a method of treatment using a reduced dose of consensus interferon and a dose of interferon-tau is provided. Consensus interferon-alpha is also known as interferon alfacon-1 (Infergen®) and is a recombinant non-naturally occurring type-1 interferon. The 166 amino acid sequence of interferon alfacon-1 was derived by scanning the sequences of several natural interferon-alpha subtypes and assigning the most frequently observed amino acid in each corresponding position. Using a WHO international potency standard for recombinant interferon alpha, the specific activity of interferon alfacon-1 in in vitro cytopathic effect assay was 1×10⁹ IU/mg.

Interferon alfacon-1 is indicated for the treatment of chronic hepatitic C infection, and specifically in patients with compensated liver disease who have anti-hepatitis C serum antibodies and/or the presence of hepatitis C RNA. Adverse effects experienced during treatment with interferon alfacon-1 include flu-like symptoms of headache, fatigue, fever, rigors, myalgia, sweating, and arthralgia. Depression is also reported and is a common cause for discontinuation of treatment.

The recommended dose of interferon alfacon-1 for treatment of hepatitis C is 9 μg three times a week administered subcutaneously as a single injection for 24 weeks.

The present method contemplates treatment with interferon alfacon-1 at a dose less than the recommended 9 μg, given three times a week via injection, in conjunction with an oral dose of interferon-tau.

### Interferon-beta1a

Interferon-beta1a (Avonex®, Rebif®) is indicated for the treatment of patients with relapsing forms of multiple sclerosis, to slow the accumulation of physical disability and decrease the frequency of clinical exacerbations. The amino acid sequence of the interferon-beta1a sold under the trade designation Rebif® is identical to that of natural fibroblast derived human interferon-beta, and is glycosylated with a single N-linked complex carbohydrate moiety.

The interferon-beta1a sold under the trade designation Rebif® is a 166 amino acid glycoprotein produced by recombinant technology in CHO cells. It has a specific activity of approximately 270×10⁶ IU/mg, as measured using a WHO natural interferon beta standard. Thus, the 44 μg dose of the interferon-beta1a Rebif® contains approximately 12×10⁶ IU of antiviral activity. The interferon-beta1a sold under the trade designation Avonex® has a specific activity of approximately 200×10⁶ international units (IU) of antiviral activity per mg of interferon beta-1a, determined specifically by an in vitro cytopathic effect bioassay using lung carcinoma cells (A549) and encephalomyocarditis virus (ECM). The 30 μg dose of this interferon-beta1a contains approximately 6×10⁶ IU of antiviral activity.

The adverse reactions most commonly reported in patients associated with the use of interferon beta-1a are flu-like and other symptoms occurring within hours to days following an injection. Symptoms can include myalgia, fever, fatigue, headaches, chills, nausea, and vomiting. Some patients have experienced paresthesias, hypertonia and myasthenia. The most frequently reported adverse reactions resulting in clinical intervention (e.g., discontinuation of treatment or the need for concomitant medication to treat an adverse reaction symptom) are flu-like symptoms and depression.

The recommended dosage of the interferon-beta1a sold under the trade designation Avonex® is 30 μg injected intramuscularly once a week. The recommended dosage of the interferon-beta1a sold under the trade designation Rebif® is 44 μg injected subcutaneously three times a week.

Accordingly, the method described herein relates to a method for treating relapsing forms of multiple sclerosis, such a relapsing-remitting multiple sclerosis, by selecting a dose of interferon-beta1a that is less than a recommended dose and administering the reduced dose to the patient along with a dose of interferon-tau. An exemplary treatment protocol is given in Example 5.

### Interferon-beta 1b

In another embodiment, the method provides a combined treatment with a dose of interferon-beta1b that is lower than the recommended dose in combination with an oral dose of interferon-tau, for treatment of patients diagnosed with relapsing forms of multiple sclerosis. Interferon-beta1b (Betaseron®) is a recombinant 165 amino acid protein, with a sequence identical to that of human interferon-beta1b other than a seine for cysteine residue substitution at position 17. The specific activity of interferon-beta1b is approximately 32×10⁶ IU/mg, as measured by comparing the activity to a WHO antiviral activity reference standard.

Multiple sclerosis patients treated with interferon-beta1b experience adverse reactions of depression, suicidal ideation and injection site necrosis. Flu-like symptoms and injection site reactions are also common.

Dosing of interferon-beta1b is recommended on an escalating dose titration schedule, where a dose of 0.0625 mg is given for weeks 1-2 of treatment, a dose of 0.125 mg is given for weeks 5-6 of treatment, a dose of 0.1875 mg is given for weeks 5-6 of treatment, and then a dose of 0.25 mg is given for week 7 and subsequent treatment weeks for an indefinite period of time.

The present method contemplates, for example, treating a patient diagnosed with relapsing remitting multiple sclerosis according to the dose titration schedule recommended in the product labeling for interferon-beta1b, except that the dose given at week 7 and for all subsequent weeks remains at 0.1875 mg and a dose of interferon-tau is provided beginning at week 7 and subsequent weeks. Alternatively, the method contemplates a treatment regimen where the doses in the interferon-beta1b dose titration schedule are reduced by 50-75% and a supplemental dose of interferon-tau is provided. Other variations can be discerned by a skilled medical provider.

It will be appreciated that the method of treatment described herein can be used when one or more additional therapeutic compounds are being administered. In this manner the described dosage forms may be used in the preparation of a medicament for administration to an individual in need of type-1 interferon treatment or undergoing type-1 interferon treatment. For example, hepatitis C is often treated using a combination of an interferon-alpha and ribavirin, a nucleoside analogue. However, the combination of ribavirin and interferon is associated with considerably more side effects than interferon treatment alone. Accordingly, it is contemplated that reducing the dosage of the interferon-alpha and/or ribavirin, and adding interferon-tau to the treatment regimen, a reduction in unwanted side effects is achieved yet efficacious therapy is maintained.

The dose of interferon-tau is preferably given via mouth, generally referred to herein as being given orally or via oral administration. When given by mouth, the dosage can be formulated to maximize oropharyngeal delivery, such as by providing a lozenge or buccal patch containing the dose of interferon-tau. A syrup or solution that is held in the mouth prior to ingestion can also be used to achieve oropharyngeal delivery. Alternatively, and in other embodiments, the dosage is formulated for delivery to the gastroinstestinal tract, as in a tablet, capsule, or pill, or in a solution that is swallowed upon placement in the oral cavity. Preparation of dosage forms for protein delivery is well known in the art and within the abilities of a skilled artisan.

### EXAMPLES

The following examples are illustrative in nature and are in no way intended to be limiting.

### Example 1

### Treatment of Chronic Hepatitis C with Interferon-alpha2b and Interferon-tau

A 34 year old male subject is diagnosed with chronic hepatitis C and is indicated for treatment with interferon-alpha2b. The subject is treated with 1×10⁶ IU of interferon-alpha2b, one-third the label dose recommended on the product label of interferon-alpha2b (Intron® A) for treatment of chronic hepatitis C. The interferon-alpha 2b is administered subcutaneously three times a week. The subject is also treated with an oral dose of interferon-tau of 2×10⁶ IU, bringing the total interferon dose to that recommended on the product label for interferon-alpha 2b. The interferon-tau is taken orally three times a week, on the same days, few hours prior to the injection, or and preferably at the same time, as the subcutaneous injection of interferon-alpha2b.

During the 16 week treatment period, the subject reports no fever, fatigue, nausea and vomiting, or gastrointestinal disorders.

### Example 2

### Treatment of Malignant Myeloma with Interferon-alpha2b and Interferon-tau

A 28 year old female subject is diagnosed with malignant myeloma and is indicated for treatment with interferon-alpha2b. The subject is treated with 20×10⁶ IU/m² of interferon-alpha2b for five consecutive days, the interferon given intravenously over 20 minutes. The patient has a severe adverse reaction of a granulocyte count of less than 500 mm³, but greater than 250 mm³. Treatment at this dosage is discontinued.

A week later, treatment is resumed using one-quarter the label dose recommended on the product label of interferon-alpha2b (Intron® A), i.e., a dose of 5×10⁶ IU/m², given intravenously. The patient is also treated with an oral dose of interferon-tau of 1×10⁸ IU taken daily. During a subsequent four week treatment period, the subject's granulocyte count remains above 500 mm³ and no adverse events are observed.

### Example 3

### Treatment of Kaposi's Sarcoma with Interferon-alpha2a and Interferon-tau

A 33 year old male subject is treated for AIDS-related Kaposi's sarcoma with a dose of 36×10⁶ IU daily for two weeks by intramuscular injection. Due to severe side effects and toxicity, treatment at this dose is discontinued. After recovery from the side effects, treatment is resumed using a dose of 1×10⁶ IU, 3×10⁶ IU, and 6×10⁶ IU each daily for three days followed by 12×10⁶ IU daily for the remainder of a 10-12 week treatment period. In addition, the patient is given an oral solution containing a dose of interferon tau of 5×10⁸ IU/day. No side effects or toxicity are reported.

### Example 4

### Treatment of Hepatitis C with Peg-Interferon-alpha2a and Interferon-tau

A 42 year old female subject is diagnosed with hepatitis C and treatment with peginterferon-alfa2a (Pegasys®) is initiated. The woman is treated with the recommended label dose of 180 μg once weekly for four weeks by subcutaneous administration in the abdomen. Weekly blood samples and analysis reveal a neutrophil count of less than 750 cells/mm³ after the fourth week of treatment. Thus, the dose of peginterferon-alfa2a is reduced to 135 μg once weekly, given by the same route of administration. The subject is additionally treated with a formulation of interferon-tau, given as a lozenge in a dose of 3×10⁵ U/day. Weekly blood samples over a subsequent six week treatment period show an increase in neutrophil count to above 1000 cells mm³ and a reduction in viral titer.

### Example 5

### Treatment of Relapsing Remitting Multiple Sclerosis with Interferon-beta1a and Interferon-tau

A 39 year old female subject is diagnosed with a relapsing form of multiple sclerosis, relapsing remitting multiple sclerosis. To slow progression of the disease and to increase the periods between relapses (a decreased frequency of relapse), the subject is given an 11 μg dose of interferon-beta1a subcutaneously three times per week and a daily oral dose of interferon-tau in an amount of 9×10⁶ IU/day.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A treatment method, comprising:
administering via injection an interferon-alpha or an interferon-beta in an amount less than a recommended dose for treatment of a disorder, said amount being insufficient for treatment of the disorder; and
administering by mouth interferon-tau in an amount effective alone or in combination with the interferon-alpha or interferon-beta for treatment of the disorder.

2. The method of claim 1, wherein said administering via injection comprises administering interferon-alpha2b.

3. The method of claim 2, wherein said administering comprises administering for treatment of a disorder selected from the group consisting of chronic hepatitis C, chronic hepatitis B, malignant myeloma, hairy cell leukemia, renal cell carcinoma, and Kaposi's sarcoma.

4. The method of claim 2, wherein said administering comprises administering pegylated interferon-alpha2b.

5. The method of claim 1, wherein said administering via injection comprises administering interferon-alpha2a.

6. The method of claim 5, wherein said administering comprises administering for treatment of a disorder selected from the group consisting of chronic hepatitis C, chronic hepatitis B, malignant myeloma, hairy cell leukemia, renal cell carcinoma, and Kaposi's sarcoma.

7. The method of claim 5, wherein said administering comprises administering pegylated interferon-alpha2a.

8. The method of claim 1, wherein said administering via injection comprises administering interferon-beta1a.

9. The method of claim 8, wherein said administering comprises administering for treatment of relapsing forms of multiple sclerosis.

10. The method of claim 1, wherein said administering via injection comprises administering interferon-beta1b.

11. The method of claim 10, wherein said administering comprises administering for treatment of relapsing forms of multiple sclerosis.

12. The method of claim 1, wherein said administering via injection comprises administering consensus interferon-alpha.

13. The method of claim 12, wherein said administering comprises administering for treatment hepatitis C.

14. An improvement in a method of treating a patient suffering from a disorder indicated for treatment with an interferon-alpha or an interferon-beta at a recommended dose; the improvement comprising:
reducing the dose of interferon-alpha or interferon-beta to less than the recommended dose; and
co-administering a dose interferon-tau, wherein the dose of interferon-alpha or interferon-beta and the dose of interferon-tau provide a combined total dose that is therapeutically effective for the disorder.

15. The improvement of claim 14, wherein said co-administering comprises co-administering interferon-tau to the oropharyngeal region.

16. The improvement of claim 14, wherein said co-administering comprises co-administering interferon-tau orally to the gastrointestinal tract.

17. The improvement of claim 14, wherein said reducing comprises reducing the dose and administering the reduced dose via injection.

18. The improvement of claim 14, wherein said reducing comprises reducing the dose of interferon-alpha2b recommended for treatment of a disorder selected from the group consisting of chronic hepatitis C, chronic hepatitis B, malignant myeloma, hairy cell leukemia, renal cell carcinoma, and Kaposi's sarcoma.

19. The improvement of claim 14, wherein said reducing comprises reducing the dose of interferon-alpha2a recommended for treatment of a disorder selected from the group consisting of chronic hepatitis C, hairy cell leukemia, AIDS-related Kaposi's sarcoma, and chronic myelogenous leukemia.

20. The improvement of claim 14, wherein said reducing comprises reducing the dose of interferon-beta1a recommended for treatment of relapsing forms of multiple sclerosis.

21. The improvement of claim 14, wherein said reducing comprises reducing the dose of interferon-beta1b recommended for treatment of relapsing forms of multiple sclerosis.

22. The improvement of claim 14, wherein said reducing comprises reducing the dose of interferon-alfacon-1 recommended for treatment of hepatitis C.

23. The improvement of claim 14, wherein said reducing comprises reducing the dose of pegylated interferon-alpha2b recommended for treatment of chronic hepatitis C.

24. The improvement of claim 14, wherein said reducing comprises reducing the dose of pegylated interferon-alpha2a recommended for treatment of chronic hepatitis C.
